(19) United States
(12) Patent Application Publication
(10) Pub. No.: US 2022/0406469 A1
(43) Pub. Date: Dec. 22, 2022

(54) INFORMATION PROCESSING SYSTEM

(71) Applicant: Hitachi, Ltd., Tokyo (JP)

(72) Inventors: Shunki NAKAGAWA, Tokyo (JP); Wataru TAKEUCHI, Tokyo (JP); Shinji TARUMI, Tokyo (JP)

(21) Appl. No.: 17/829,708

(22) Filed: Jun. 1, 2022

(30) Foreign Application Priority Data
Jun. 22, 2021 (JP) 2021-102999

(51) Int. Cl. G16H 50/50; G16H 50/70

(52) U.S. Cl. CPC G16H 50/50; G16H 50/70

(57) ABSTRACT

An object of the invention is to improve prediction accuracy of data including a low-frequency medical treatment practice. Provided is an information processing system including: a transition information generation unit configured to generate transition information of an event for each patient of a plurality of patients based on medical information of the plurality of patients; a medical treatment process classification unit configured to classify a process of a medical treatment practice included in the event into a plurality of groups based on a threshold value related to a frequency of the medical treatment practice according to the generated transition information; a medical treatment process granularity adjustment unit configured to aggregate items of the process of the medical treatment practice in at least a part of the groups; a prediction model generation unit configured to generate a prediction model for each of the groups; and an output unit configured to classify input data of medical information of a new patient into any one of the groups, and to output an occurrence of an event of the new patient using the prediction model.

### CLAIM OF PRIORITY

The present application claims priority from Japanese patent application JP 2021-102999 filed on Jun. 22, 2021, the content of which is hereby incorporated by reference into this application.

### BACKGROUND OF THE INVENTION

### 1. Field of the Invention

The present invention relates to an information processing system that processes medical information.

### 2. Description of the Related Art

In recent years, machine learning for medical information including a patient state, a medical treatment practice, and the like is used to predict an occurrence of an event such as life-or-death of a patient, and a medical treatment of a doctor is supported based on a result of the prediction.

US 2014-0058738 discloses that a medical treatment of a doctor is supported by selecting a plurality of processes of the medical treatment practice from a clinical guideline, generating a prediction model for each selected process of the medical treatment practice, calculating a probability of a patient outcome, and presenting the calculated probability to the doctor.

The medical information described above may include a low-frequency medical treatment practice due to diversification and complication of the medical treatment associated with the development of precision medicine. When the machine learning is used for such data, learning is also performed based on data having low correlation with an objective variable, and sufficient prediction accuracy may not be obtained.

Therefore, in utilizing the medical information for medical treatment support, there is a problem of improving prediction accuracy with data including the low-frequency medical treatment practice, but a method thereof is not considered in US 2014-0058738.

### SUMMARY OF THE INVENTION

In order to solve the above problems, the invention provides an information processing system that processes medical information, the information processing system including: a transition information generation unit configured to generate transition information of an event for each patient of a plurality of patients based on medical information of the plurality of patients; a medical treatment process classification unit configured to classify a process of a medical treatment practice included in the event into a plurality of groups based on a threshold value related to a frequency of the medical treatment practice according to the transition information; a medical treatment process granularity adjustment unit configured to aggregate items of the process of the medical treatment practice in at least a part of the groups; a prediction model generation unit configured to generate a prediction model for each of the groups; and an output unit configured to classify input data of medical information of a new patient into any one of the groups, and to output an occurrence of an event of the new patient using the prediction model.

According to the invention, the occurrence of the event can be predicted with high accuracy even based on data including low-frequency medical treatment practice by dividing the process of the medical treatment practice into the plurality of groups, adjusting a granularity of the item of the process of the medical treatment practice so as to satisfy a frequency required in machine learning, and generating the prediction model for each group.

### BRIEF DESCRIPTION OF THE DRAWINGS

FIG. 1 is a block diagram illustrating a hardware configuration of an information processing system according to a first embodiment.

FIG. 2 is a table illustrating a configuration of data of patient information stored in a patient information storage unit of the system according to the first embodiment.

FIG. 3 is a table illustrating a configuration of data of examination information stored in an examination information storage unit of the system according to the first embodiment.

FIG. 4 is a table illustrating a configuration of data of diagnostic information stored in a diagnostic information storage unit of the system according to the first embodiment.

FIG. 5 is a table illustrating a configuration of data of medical treatment information stored in a medical treatment information storage unit of the system according to the first embodiment.

FIG. 6 is a table illustrating a configuration of data of dictionary information stored in a dictionary information storage unit of the system according to the first embodiment.

FIG. 7 is a flowchart of analysis target person extraction processing of the system according to the first embodiment.

FIG. 8 is a table illustrating objective variable information generated in objective variable generation processing of the system according to the first embodiment.

FIG. 9 is a flowchart of transition information generation processing of the system according to the first embodiment.

FIG. 10 is a table illustrating transition information generated in the transition information generation processing of the system according to the first embodiment.

FIG. 11 is a flowchart of medical treatment process classification processing of the system according to the first embodiment.

FIG. 12 is a table illustrating a frequency of a process of a medical treatment practice to be calculated in the medical treatment process classification processing of the system according to the first embodiment.

FIG. 13 is a flowchart of medical treatment process granularity adjustment processing of the system according to the first embodiment.

FIG. 14 is a flowchart of prediction model generation processing of the system according to the first embodiment.

FIG. 15 is a flowchart of output processing of the system according to the first embodiment.

FIG. 16 is a diagram illustrating a medical treatment support screen to be output in the output processing of the system according to the first embodiment.

FIG. 17 is a flowchart of medical treatment process classification processing of a system according to a second embodiment.

FIG. 18 is a table illustrating a frequency and an average information amount in a process of a medical treatment practice to be calculated in the medical treatment process classification processing of the system according to the second embodiment.

### DESCRIPTION OF THE PREFERRED EMBODIMENTS

Hereinafter, embodiments of the invention will be sequentially described with reference to the drawings.

### First Embodiment

A first embodiment is an embodiment of an information processing system that processes medical information, and the information processing system includes: a transition information generation unit configured to generate transition information of an event for each patient of a plurality of patients based on medical information of the plurality of patients; a medical treatment process classification unit configured to classify a process of a medical treatment practice included in the event into a plurality of groups based on a threshold value related to a frequency of the medical treatment practice according to the transition information; a medical treatment process granularity adjustment unit configured to aggregate items of the process of the medical treatment practice in at least a part of the groups; a prediction model generation unit configured to generate a prediction model for each of the groups; and an output unit configured to classify input data of medical information of a new patient into any one of the groups, and to output an occurrence of an event of the new patient using the prediction model.

That is, in the information processing system of the present embodiment, the process of the medical treatment practice is divided into the plurality of groups based on the threshold value related to the frequency, a granularity of the items in the process of the medical treatment practice in the group that does not satisfy the threshold value is aggregated, and the prediction model is generated for each group. Accordingly, the granularity of the items in the process of the medical treatment practice is adjusted so as to satisfy the frequency required in the machine learning, and the occurrence of the event can be predicted with high accuracy.

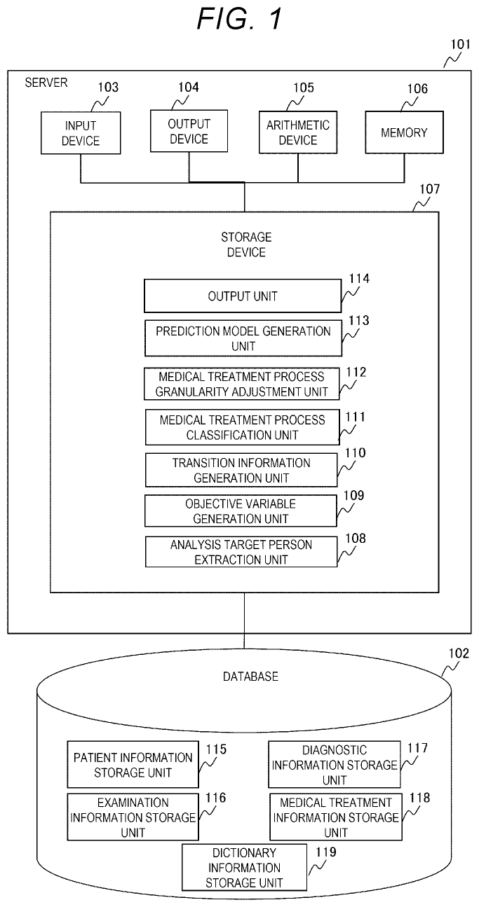

FIG. 1 is a block diagram illustrating a hardware configuration of the information processing system according to the first embodiment. The information processing system includes a server 101 and a database 102. The server 101 and the database 102 are connected to each other such that the server 101 can access data stored in the database 102.

The server 101 is a computer including an input device 103, an output device 104, an arithmetic device 105 that executes a program, and a memory 106 and a storage device 107 that store a program. The input device 103 is a mouse, a keyboard, or the like, and is an interface through which an input to the server 101 is received. The output device 104 is a display device, a printer, or the like, and outputs a calculation result of the arithmetic device 105.

The arithmetic device 105 is a CPU, a GPU, or the like, and executes a program loaded in the memory 106. The memory 106 includes a ROM, which is a non-volatile storage element, and a RAM, which is a volatile storage element. The ROM stores an invariable program (for example, a BIOS) and the like. The RAM is a high-speed volatile storage element such as a dynamic random access memory (DRAM), and temporarily stores a program stored in the storage device 107 and data to be used when the program is executed. The storage device 107 is a non-volatile storage device such as a magnetic storage device (HDD) or a flash memory (SSD), and stores a program to be executed by the arithmetic device 105 and data to be used when the program is executed.

Specifically, the storage device 107 stores programs for implementing units of an analysis target person extraction unit 108, an objective variable generation unit 109, a transition information generation unit 110, a medical treatment process classification unit 111, a medical treatment process granularity adjustment unit 112, a prediction model generation unit 113, and an output unit 114.

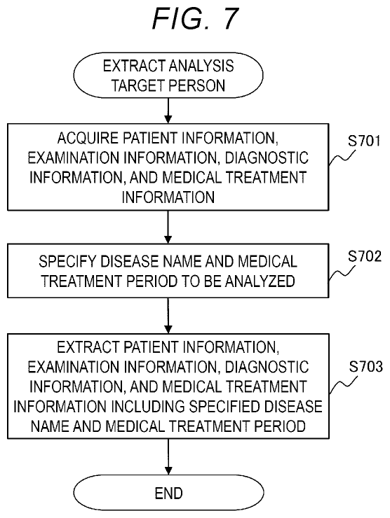

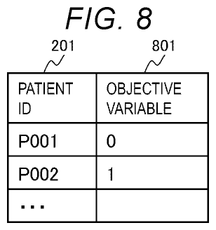

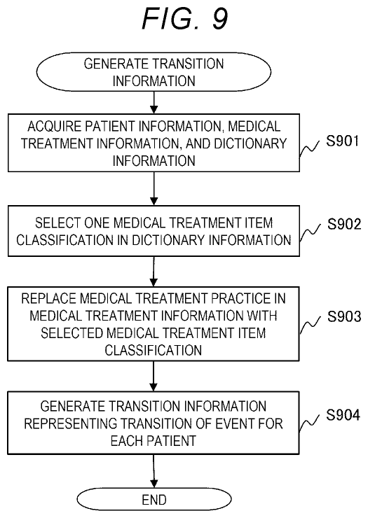

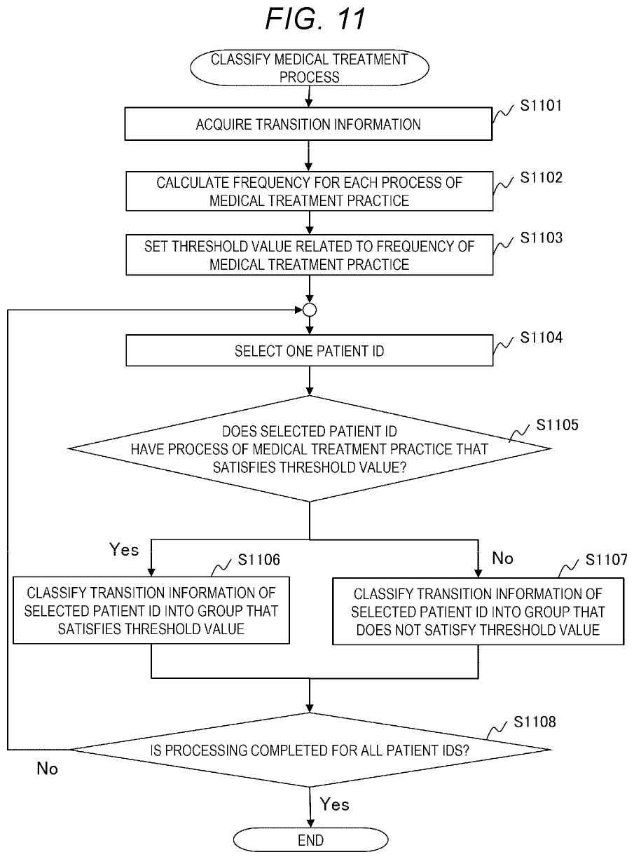

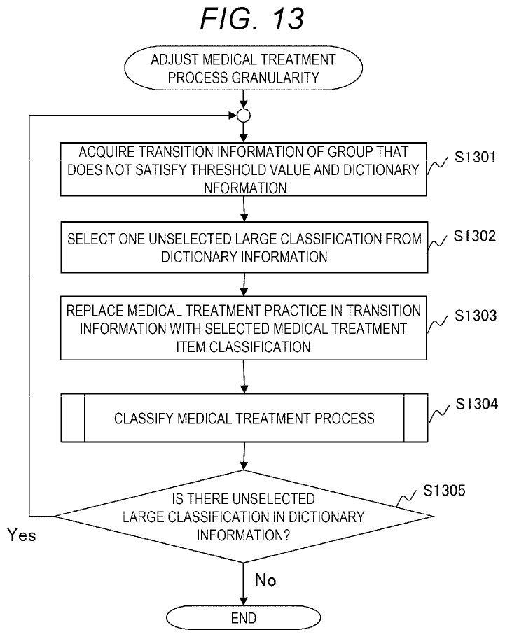

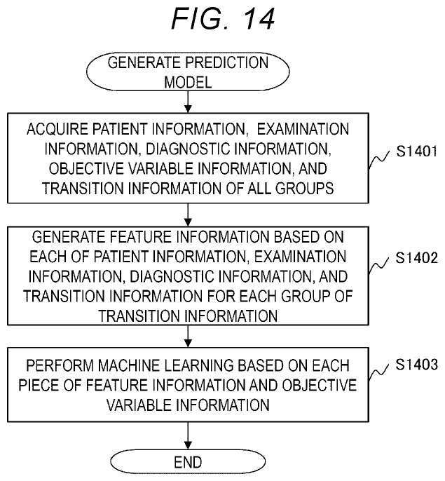

The analysis target person extraction unit 108 extracts an analysis target person by executing a predetermined program (see FIG. 7). The objective variable generation unit 109 generates an objective variable for each patient by executing a predetermined program (see FIG. 8). The transition information generation unit 110 generates transition information of an event for each patient by executing a predetermined program (see FIG. 9). The medical treatment process classification unit 111 classifies, by executing a predetermined program, a process of a medical treatment practice included in the event into a plurality of groups based on a threshold value related to a frequency of the medical treatment practice (see FIG. 11). The medical treatment process granularity adjustment unit 112 adjusts, by executing a predetermined program, granularity of items in the process of the medical treatment practice in a group that does not satisfy the threshold value (see FIG. 13). The prediction model generation unit 113 generates a prediction model for each classified group by executing a predetermined program (see FIG. 14).

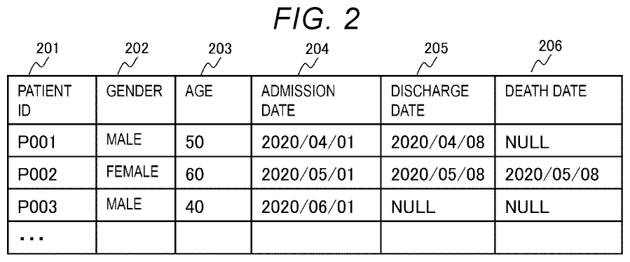

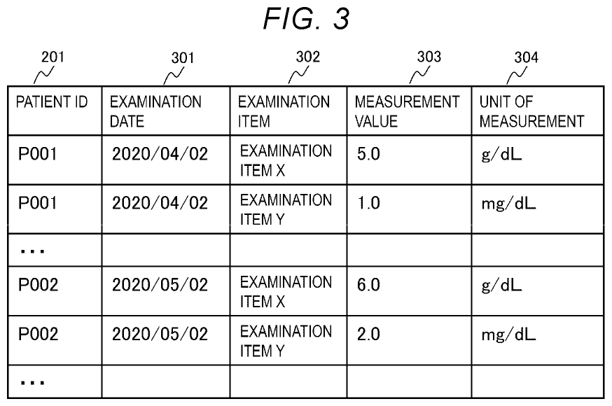

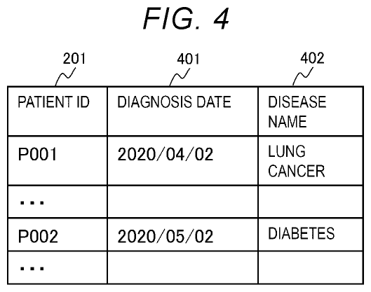

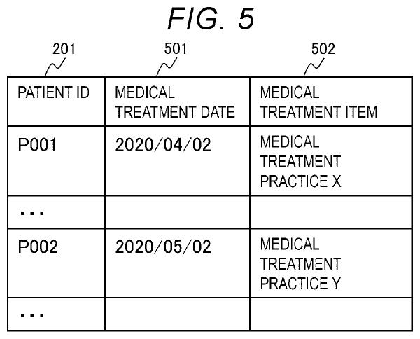

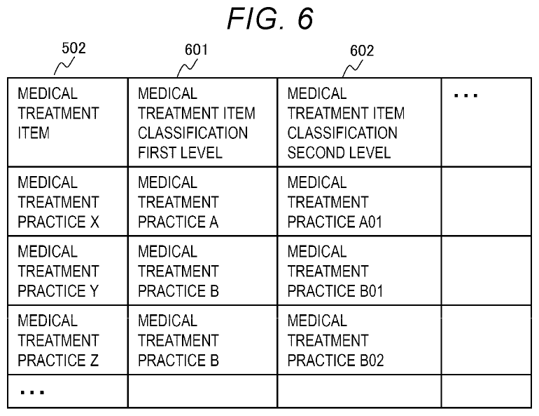

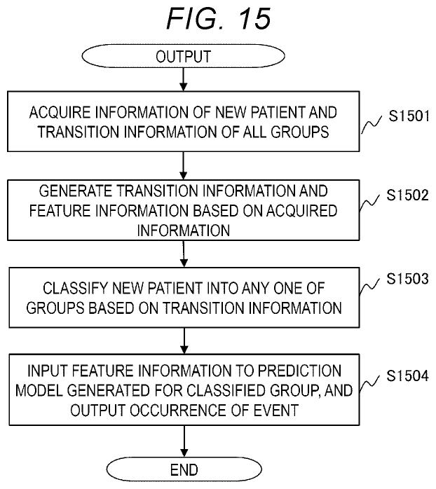

The output unit 114 classifies input data of a new patient into any one of the groups by executing a predetermined program, and outputs an occurrence of the event of the new patient by using the prediction model (see FIG. 15). The database 102 stores data for the server 101 to analyze the medical information, that is, a patient information storage unit 115 (see FIG. 2), an examination information storage unit 116 (see FIG. 3), a diagnostic information storage unit 117 (see FIG. 4), a medical treatment information storage unit 118 (see FIG. 5), and a dictionary information storage unit 119 (see FIG. 6).

FIG. 2 is a table illustrating a configuration of patient information stored in the patient information storage unit 115 according to the first embodiment. The patient information includes data of a patient ID 201, a gender 202, an age 203, an admission date 204, a discharge date 205, and a death date 206.

The patient ID 201 is an identifier for uniquely identifying a patient. The gender 202 is a gender of the patient. The age 203 is an age of the patient. The admission date 204 is a date when the patient was admitted. The discharge date 205 is a date when the patient was discharged. When the patient is not discharged, NULL is assigned. The death date 206 is a date when the patient died. When the patient is not dead, NULL is assigned.

FIG. 3 is a table illustrating a configuration of examination information stored in the examination information storage unit 116 of the system according to the first embodiment. The examination information includes data of the patient ID 201, an examination date 301, an examination item 302, a measurement value 303, and a unit of measurement 304. The examination date 301 is a date when a doctor performed an examination. The examination item 302 is an item of the examination. The measurement value 303 is a measurement value of the examination item 302. The unit of measurement 304 is a unit of measurement of the examination item 302.

FIG. 4 is a table illustrating a configuration of diagnostic information stored in the diagnostic information storage unit 117 of the system according to the first embodiment. The diagnostic information includes data of the patient ID 201, a diagnosis date 401, and a disease name 402. The diagnosis date 401 is a date when a doctor made a diagnosis of a disease for the patient. The disease name 402 is a name of the disease.

FIG. 5 is a table illustrating a configuration of medical treatment information stored in the medical treatment information storage unit 118 according to the first embodiment. The medical treatment information includes data of the patient ID 201, a medical treatment date 501, and a medical treatment item 502. The medical treatment date 501 is a date when a doctor performed a medical treatment. The medical treatment item 502 is an item of the medical treatment practice.

FIG. 6 is a table illustrating a configuration of dictionary information stored in the dictionary information storage unit 119 of the system according to the first embodiment. The dictionary information includes data of the medical treatment item 502, a medical treatment item classification first level 601, and a medical treatment item classification second level 602.

Each of the medical treatment item classification first level 601 and the medical treatment item classification second level 602 is a classification of the medical treatment item 502, and the same classification is assigned to the medical treatment items 502 in which a site, a working capacity, and the like belong to the same system. For example, when the medical treatment item 502 is a medical treatment practice related to diabetes such as insulin injection, based on an anatomical therapeutic chemical classification system (ATC classification) created by the World Health Organization, the medical treatment item classification first level 601 is assigned an “A digestive tract and metabolic effect”, and the medical treatment item classification second level 602 is assigned an “A10 diabetic medicine”, which is a next largest classification of the medical treatment item classification first level 601.

FIG. 7 is a flowchart of an analysis target person extraction processing of the system according to the first embodiment. The analysis target person extraction processing is executed by the analysis target person extraction unit 108 of the server 101.

First, the patient information, the examination information, the diagnostic information, and the medical treatment information are acquired (S701). The patient information is acquired from the patient information storage unit 115. The examination information is acquired from the examination information storage unit 116. The diagnostic information is acquired from the diagnostic information storage unit 117. The medical treatment information is acquired from the medical treatment information storage unit 118.

Next, a disease name and a medical treatment period to be analyzed are specified from the acquired diagnostic information (S702), patient information, examination information, diagnostic information, and medical treatment information including the specified disease name and medical treatment period are extracted (S703), and the processing is ended.

FIG. 8 is a table illustrating a configuration of objective variable information generated in objective variable generation processing of the system according to the first embodiment. The objective variable generation processing is executed by the objective variable generation unit 109 of the server 101. An objective variable 801 is an objective variable representing an event to be predicted. For example, in a case where a discharge or a death of a patient is to be predicted, if the death date is NULL for a discharged patient, 0 is assigned as an objective variable representing the discharge, and if the death date is not NULL, 1 is assigned as an objective variable representing the death.

FIG. 9 is a flowchart of transition information generation processing of the system according to the first embodiment. The transition information generation processing is executed by the transition information generation unit 110 of the server 101. First, the patient information, the medical treatment information, and the dictionary information are acquired (S901). The patient information and the medical treatment information are extracted by the analysis target person extraction processing (FIG. 7). The dictionary information is acquired from the dictionary information storage unit 119.

Next, one medical treatment item classification in the dictionary information is selected (S902), and is used to replace a medical treatment practice in the medical treatment information (S903). Next, transition information representing transition of an event for each patient is generated based on the acquired patient information and medical treatment information (S904), and the processing is ended.

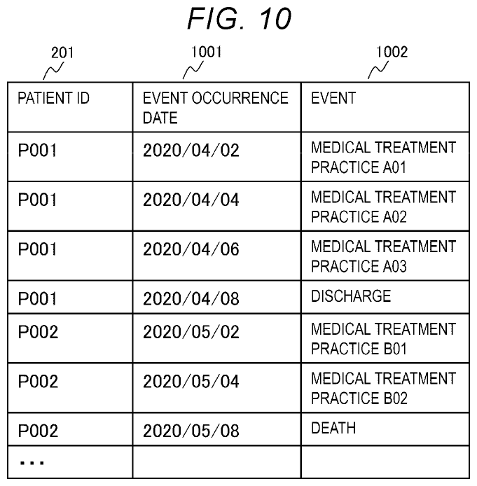

FIG. 10 is the transition information generated in step S904 of FIG. 9. An event occurrence date 1001 and an event 1002 respectively represent an occurrence date of an event happening to each patient and a content of the event. For example, FIG. 10 shows the transition information that is generated in a case where the discharge and the death in the patient information and the medical treatment practice in the medical treatment information are regarded as events, and replacement by the medical treatment item classification second level 602 is performed. Accordingly, the transition of the event can be confirmed for each patient.

FIG. 11 is a flowchart of medical treatment process classification processing of the system according to the first embodiment. The medical treatment process classification processing is executed by the medical treatment process classification unit 111 of the server 101.

First, the transition information is acquired (S1101). The transition information is generated by the transition information generation processing (FIG. 9). Next, a frequency is calculated for each process of a medical treatment practice (S1102).

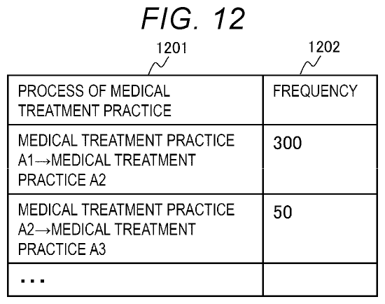

FIG. 12 shows the frequency of the process of the medical treatment practice calculated in step S1102 of FIG. 11. A process 1201 of the medical treatment practice is a process of two consecutive medical treatment practices in the transition information. A frequency 1202 is the frequency of the patient in the process 1201 of the medical treatment practice.

Next, a threshold value related to the frequency of the medical treatment practice is set (S1103). Next, one patient ID is selected (S1104), and it is determined whether the selected patient ID has a process of the medical treatment practice that satisfies the threshold value (S1105). As a result, if the selected patient ID has the process of the medical treatment practice that satisfies the threshold value, the transition information of the selected patient ID is classified into a group that satisfies the threshold value (S1106). On the other hand, if the selected patient ID does not have the process of the medical treatment practice that satisfies the threshold value, the transition information of the selected patient ID is classified into a group that does not satisfy the threshold value (S1107). For example, when 200 is set as the threshold value related to the frequency, the transition information of the patient ID that has the process of the medical treatment practice having a frequency of 200 or more is classified into the group that satisfies the threshold value. Accordingly, it is possible to extract the transition information of the patient having the process of the medical treatment practice that satisfies a frequency required in the machine learning.

Next, it is determined whether the processing is completed for all patient IDs (S1108). As a result, if the processing is not completed for a part of the patient IDs, the processing returns to step S1104 and a next patient ID is selected. On the other hand, if the processing is completed for all the patient IDs, this processing is ended.

FIG. 13 is a flowchart of medical treatment process granularity adjustment processing of the system according to the first embodiment. The medical treatment process granularity adjustment processing is executed by the medical treatment process granularity adjustment unit 112 of the server 101.

First, the transition information of the group that does not satisfy the threshold value and the dictionary information are acquired (S1301). The transition information of the group that does not satisfy the threshold value is acquired from the medical treatment process classification processing (FIG. 11). The dictionary information is acquired from the dictionary information storage unit 119.

Next, one unselected large classification is selected from among medical treatment item classifications in the dictionary information (S1302), and is used to replace the medical treatment practice in the transition information (S1303). For example, when the medical treatment item classification second level 602 in the dictionary information is selected in previous processing, the medical treatment item classification first level 601 is selected in step S1302, and replacement by the medical treatment item classification first level 601 is performed in S1303. Accordingly, it is possible to increase the frequency of each process of the medical treatment practice, for the transition information that is classified, in the medical treatment process classification processing, into the group that does not satisfy the threshold value.

Next, the medical treatment process classification processing is executed (S1304), and it is determined whether there is an unselected large classification in the medical treatment item classifications of the dictionary information (S1305). As a result, if there is an unselected large classification, the processing returns to step S1301, and the transition information, extracted in step S1304, of the group that does not satisfy the threshold value is acquired. On the other hand, if there is no unselected large classification, the processing is ended.

FIG. 14 is a flowchart of prediction model generation processing of the system according to the first embodiment. The prediction model generation processing is executed by the prediction model generation unit 113 of the server 101.

First, the patient information, the examination information, the diagnostic information, the objective variable information, and the transition information of all the groups are acquired (S1401). The patient information, the examination information, and the diagnostic information are extracted by the analysis target person extraction processing (FIG. 7). The objective variable information is generated by the objective variable generation processing. The transition information of all the groups is acquired from the medical treatment process classification processing (FIG. 11) and the medical treatment process granularity adjustment processing (FIG. 13).

Next, for each group of the acquired transition information, feature information is generated based on each of the patient information, the examination information, the diagnostic information, and the transition information (S1402), the machine learning is performed based on the feature information and the objective variable information (S1403), and then the processing is ended. At this time, a different machine learning algorithm may be used for each group of the transition information. Specifically, overlearning can be prevented, by changing complexity of the machine learning algorithm in view of the frequency of the process of the medical treatment practice.

FIG. 15 is a flowchart of output processing of the system according to the first embodiment. This output processing is executed by the output unit 114 of the server 101.

First, information of a new patient and the transition information of all the groups are acquired (S1501). The information of the new patient is input from the input device 103. The transition information of all the groups is acquired from the medical treatment process classification processing (FIG. 11) and the medical treatment process granularity adjustment processing (FIG. 13).

Next, transition information and feature information are generated from the acquired information (S1502), and the new patient is classified into any one of the groups based on the transition information (S1503). Accordingly, an appropriate prediction model can be selected according to a type of the process of the medical treatment practice. Next, the feature information is input to the prediction model generated for the classified group, an occurrence of an event of the new patient is output (S1504), and then this processing is ended.

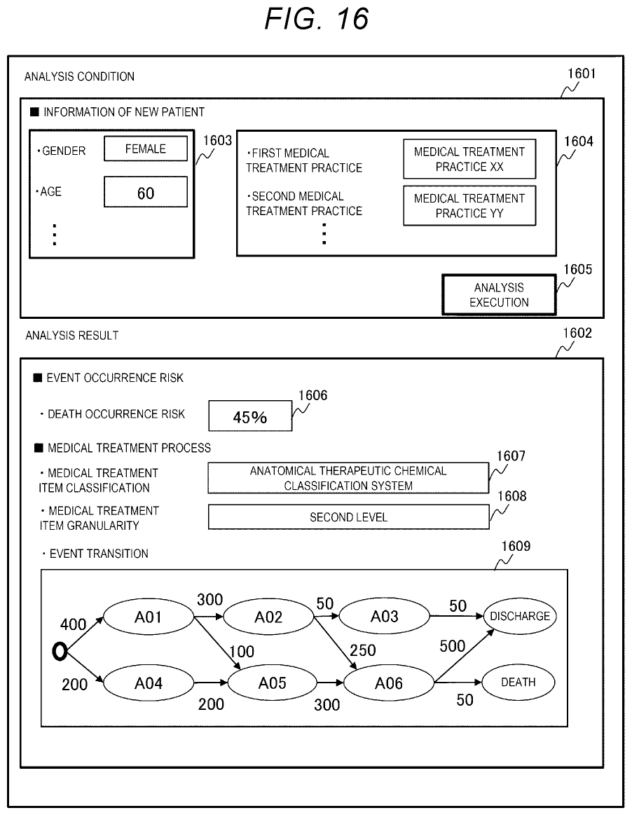

FIG. 16 is a medical treatment support screen to be output in the output processing of the system according to the first embodiment. The medical treatment support screen includes an analysis condition area 1601 and an analysis result area 1602.

The analysis condition area 1601 includes an input area 1603 of the feature information, an input area 1604 of the transition information, and an analysis execution button 1605. When the new patient inputs the information to the input area 1603 of the feature information and the input area 1604 of the transition information and clicks the analysis execution button 1605, the output processing can be executed.

The analysis result area 1602 includes a death occurrence risk 1606, a medical treatment item classification 1607, a medical treatment item granularity 1608, and an event transition 1609, and is displayed by clicking the analysis execution button 1605.

The death occurrence risk 1606 is a probability of an occurrence of an event output from an executed machine learning model. Accordingly, the death occurrence risk of the new patient can be displayed.

The medical treatment item classification 1607 and the medical treatment item granularity 1608 indicates names of the dictionary information and the medical treatment item classification described with reference to FIG. 6. Here, a case where the anatomical therapeutic chemical classification system is selected for the dictionary information and the medical treatment item classification second level is selected for the medical treatment item classification is illustrated as an example. Accordingly, it is possible to confirm the granularity of an item of the process of the medical treatment practice displayed in the event transition 1609 to be described later.

The event transition 1609 is an example of visualization of the transition information of a group to which the executed machine learning model belongs. By displaying the transition and the frequency of events, it is possible to easily confirm a difference in medical treatment records caused due to the process of the medical treatment practice.

As described above, in the system of the first embodiment, the process of the medical treatment practice is divided into the plurality of groups based on the threshold value related to the frequency, the granularity of the items in the process of the medical treatment practice in the group that does not satisfy the threshold value is aggregated, and the prediction model is generated for each group. Accordingly, the granularity of the items in the process of the medical treatment practice is adjusted so as to satisfy the frequency required in the machine learning, and the occurrence of the event can be predicted with high accuracy.

### Second Embodiment

In a system according to a second embodiment, a process of a medical treatment practice is divided into a plurality of groups based on threshold values related to a frequency and an average information amount (entropy), granularity of an item of the process of the medical treatment practice in a group that does not satisfy the threshold values is aggregated, and a prediction model is generated for each group. Accordingly, the granularity of the item of the process of the medical treatment practice can be adjusted so as to satisfy the frequency required in machine learning and to reduce uncertainty relating to an objective variable, and an occurrence of an event can be predicted with high accuracy.

A hardware configuration of the information processing system according to the second embodiment is the same as that of the information processing system according to the first embodiment described above, and thus a description thereof will be omitted. Analysis target person extraction processing, objective variable generation processing, and transition information generation processing of the system according to the second embodiment are the same as those of the system according to the first embodiment described above, and thus a description thereof will be omitted.

In the analysis target person extraction processing of the system according to the second embodiment, an analysis target person is extracted. In the objective variable generation processing of the system according to the second embodiment, an objective variable is generated for each patient. In the transition information generation processing of the system according to the second embodiment, transition information of an event is generated for each patient.

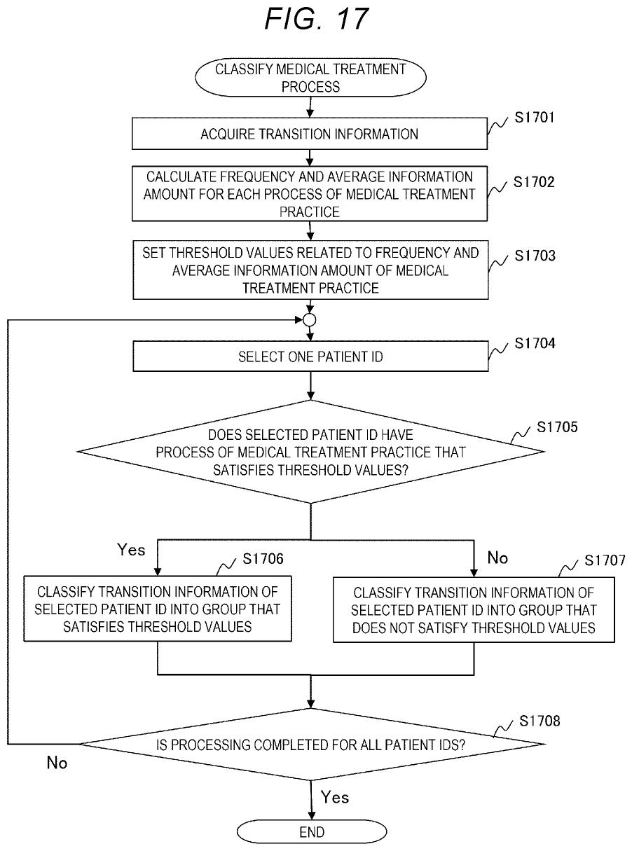

FIG. 17 is a flowchart of medical treatment process classification processing of the system according to the second embodiment. The medical treatment process classification processing is executed by the medical treatment process classification unit 111 of the server 101.

First, transition information is acquired (S1701). The transition information is generated by the transition information generation processing (FIG. 9). Next, a frequency and an average information amount are calculated for each process of the medical treatment practice (S1702).

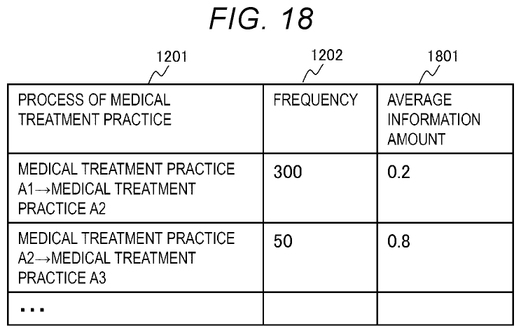

FIG. 18 shows the frequency and the average information amount of the process of the medical treatment practice calculated in step S1702 in FIG. 17. An average information amount 1801 is calculated based on a proportion of discharged patients and a proportion of dead patients in the process 1201 of the medical treatment practice. When all the patients are discharged or dead, the average information amount 1801 is 0, and when the proportion of the discharged patients and the proportion of the dead patients are equal to each other, the average information amount 1801 is 1. Accordingly, the uncertainty of the discharge or the death of the patient in the process of the medical treatment practice can be quantified. Next, threshold values related to the frequency and the average information amount of the medical treatment practice are set (S1703).

Next, one patient ID is selected (S1704), and it is determined whether the selected patient ID has a process of the medical treatment practice that satisfies the threshold values (S1705). As a result, if the selected patient ID has a process of the medical treatment practice that satisfies the threshold values, transition information of the selected patient ID is classified into a group that satisfies the threshold values (S1706). On the other hand, if the selected patient ID does not have a process of the medical treatment practice that satisfies the threshold values, the transition information of the selected patient ID is classified into a group that does not satisfy the threshold values (S1707). For example, when 200 and 0.4 are set as the threshold values related to the frequency and the average information amount, respectively, the transition information of the patient ID having the process of the medical treatment practice, which has a frequency of 200 or more and an average information amount of 0.4 or less, is classified into the group that satisfies the threshold values. Accordingly, it is possible to extract the transition information of the patient having the process of the medical treatment practice that satisfies the frequency required in the machine learning and with which the discharge or the death of the patient can be easily predicted.

Next, it is determined whether the processing is completed for all the patient IDs (S1708). As a result, if the processing is not completed for a part of the patient IDs, the processing returns to step S1704, and a next patient ID is selected. On the other hand, if the processing is completed for all the patient IDs, this processing is ended.

Medical treatment process granularity adjustment processing, prediction model generation processing, and output processing of the system according to the second embodiment are the same as those of the system according to the first embodiment described above, and thus a description thereof will be omitted. In the medical treatment process granularity adjustment processing of the system according to the second embodiment, the granularity of the item of the process of the medical treatment practice in the group that does not satisfy the threshold values is adjusted.

In the prediction model generation processing of the system according to the second embodiment, a prediction model is generated for each classified group. In the output processing of the system according to the second embodiment, input data of a new patient is classified into any one of the groups, and an occurrence of an event of the new patient is output using the prediction model.

As described above, in the system of the second embodiment, the process of the medical treatment practice is divided into the plurality of groups based on the threshold values related to the frequency and the average information amount, the granularity of the item of the process of the medical treatment practice in the group that does not satisfy the threshold values is aggregated, and the prediction model is generated for each group. Accordingly, the granularity of the item of the process of the medical treatment practice can be adjusted so as to satisfy the frequency required in the machine learning and to reduce the uncertainty relating to the objective variable, and the occurrence of the event can be predicted with high accuracy.

In the systems of the first embodiment and the second embodiment, as the medical treatment process classification processing, the frequency and the average information amount of the process of two consecutive medical treatment practices in the transition information are calculated, and alternatively the frequency and the average information amount of the process of all the consecutive medical treatment practices in the transition information may be calculated. Accordingly, in the process of all the medical treatment practices, it is possible to extract the transition information of the patient having the process of the medical treatment practice that satisfies the frequency required in the machine learning and with which the discharge or the death of the patient can be easily predicted.

In the systems of the first embodiment and the second embodiment, as the medical treatment process classification processing, the threshold values related to the frequency and the average information amount of the medical treatment practice are set, and alternatively an optimum threshold value may be selected from predetermined threshold value candidates. For example, the process of the medical treatment practice may be vectorized, an average value of a similarity between the processes of the medical treatment practice in the group that satisfies the threshold value may be calculated for each threshold value candidate, and a threshold value corresponding to a largest average value of the similarity may be selected. Accordingly, the threshold value can be optimized such that homogeneous transition information in which the similarity of the processes of the medical treatment practice is high is extracted.

In the systems of the first embodiment and the second embodiment, as the medical treatment process classification processing, the transition information is classified by determining whether the selected patient ID has the process of the medical treatment practice that satisfies the threshold value, and alternatively another method may be used. For example, the transition information may be classified by setting a threshold value related to a parameter of a process mining algorithm such as a heuristic minor, and extracting a process of a medical treatment practice having a potentially strong causal relationship. Accordingly, in the prediction model generation processing, the machine learning can be performed based on data having a high correlation with the objective variable.

What is claimed is:

1. An information processing system that processes medical information, comprising:
a transition information generation unit configured to generate transition information of an event for each patient of a plurality of patients based on medical information of the plurality of patients;
a medical treatment process classification unit configured to classify a process of a medical treatment practice included in the event into a plurality of groups based on a threshold value related to a frequency of the medical treatment practice according to the transition information;
a medical treatment process granularity adjustment unit configured to aggregate items of the process of the medical treatment practice in at least a part of the groups;
a prediction model generation unit configured to generate a prediction model for each of the groups; and
an output unit configured to classify input data of medical information of a new patient into any one of the groups, and to output an occurrence of an event of the new patient using the prediction model.

2. The information processing system according to claim 1, wherein
the prediction model generation unit changes a generation method of the prediction model for each group based on the threshold value related to the frequency.

3. The information processing system according to claim 1, wherein
the medical treatment process classification unit classifies the process of the medical treatment practice included in the event into the plurality of groups based on threshold values related to the frequency and an average information amount of the medical treatment practice according to the transition information.

4. The information processing system according to claim 2, wherein
the medical treatment process classification unit classifies the process of the medical treatment practice included in the event into the plurality of groups based on threshold values related to the frequency and an average information amount of the medical treatment practice according to the transition information.

5. The information processing system according to claim 1, wherein
the output unit classifies the input data of the medical information of the new patient into any one of the groups, and outputs the occurrence of the event of the new patient using the prediction model.

6. The information processing system according to claim 2, wherein
the output unit classifies the input data of the medical information of the new patient into any one of the groups, and outputs the occurrence of the event of the new patient using the prediction model.

7. The information processing system according to claim 3, wherein
the output unit classifies the input data of the medical information of the new patient into any one of the groups, and outputs the occurrence of the event of the new patient using the prediction model.

8. The information processing system according to claim 1, wherein
the output unit classifies the input data of the medical information of the new patient into any one of the groups, and outputs the transition information of the groups using the prediction model.

9. The information processing system according to claim 2, wherein
the output unit classifies the input data of the medical information of the new patient into any one of the groups, and outputs the transition information of the groups using the prediction model.

10. The information processing system according to claim 3, wherein
the output unit classifies the input data of the medical information of the new patient into any one of the groups, and outputs the transition information of the groups using the prediction model.

11. The information processing system according to claim 4, wherein
the output unit classifies the input data of the medical information of the new patient into any one of the groups, and outputs the transition information of the groups using the prediction model.

12. The information processing system according to claim 1, further comprising:
an analysis target person extraction unit configured to extract medical information including a disease name and a medical treatment period specified from the medical information of the patient.

13. The information processing system according to claim 2, further comprising:
an analysis target person extraction unit configured to extract medical information including a disease name and a medical treatment period specified from the medical information of the patient.

14. The information processing system according to claim 3, further comprising:
an analysis target person extraction unit configured to extract medical information including a disease name and a medical treatment period specified from the medical information of the patient.

15. The information processing system according to claim 4, further comprising:
an analysis target person extraction unit configured to extract medical information including a disease name and a medical treatment period specified from the medical information of the patient.

16. The information processing system according to claim 5, further comprising:
an analysis target person extraction unit configured to extract medical information including a disease name and a medical treatment period specified from the medical information of the patient.
